(19) United States
(12) Patent Application Publication
(10) Pub. No.: US 2026/0057963 A1
(43) Pub. Date: Feb. 26, 2026

(54) PREDICTIVE PESTICIDE RESISTANCE INFORMATION GENERATION AND USE

(71) Applicant: PIONEER HI-BRED INTERNATIONAL, INC., JOHNSTON, IA (US)

(72) Inventors: ANANTA ACHARYA, JOHNSTON, IA (US); JEAN-LUC GENET, ANDOLSHEIM (FR); CHARLOTTE HARRIS, DES MOINES, IA (US); MAMADOU MBOUP, COLMAR (FR); FRANK SCHNIEDER, LEHMAUHLEN (DE); GERARDUS W.A.M. VAN DER HEIJDEN, JOHNSTON, IA (US); KOSTE YADETA, JOHNSTON, IA (US)

(73) Assignee: PIONEER HI-BRED INTERNATONAL, INC., JOHNSTON, IA (US)

(21) Appl. No.: 18/860,291

(22) PCT Filed: Apr. 19, 2023
(86) PCT No.: PCT/US23/65942

§ 371 Date: Oct. 25, 2024

Related U.S. Application Data
(60) Provisional application No. 63/334,995, filed on Apr. 26, 2022.

(51) Int. Cl. G16B 20/20; C12Q 1/6895; G06Q 50/02; G06T 7/00; G16B 40/00

(52) U.S. Cl. CPC G16B 20/20; C12Q 1/6895; G06Q 50/02; G06T 7/0012; G16B 40/00; C12Q 2600/158; G06T 2207/10036; G06T 2207/30188

(57) ABSTRACT

The present disclosure relates to methods for management of pests, and in greater detail, to the generation of a pesticide resistance information, and to use of the pesticide resistance information in the generation of a recommended treatment protocol for a crop infested with a pest. In some embodiments, the information may be in the form of a map. The disclosure also relates to a method of predicting resistance to pesticides. The disclosure involves collection and use of genotypic sequence information of pests and genotyping, in combination with remote sensing data, to identify pesticide resistance factors.

### CROSS REFERENCE TO RELATED APPLICATIONS

This application claims priority under 35 U.S.C. section 119 to U.S. Provisional Patent Application No. 63/334,995 filed Apr. 26, 2022, which application is hereby incorporated by reference in its entirety.

### BACKGROUND

The present disclosure relates to methods for management of pests, and in greater detail, to the generation of pesticide resistance information, and to use of the pesticide resistance information in the generation of a recommended treatment protocol for a crop infested with a pest. The pesticide resistance information may be in the form of a map. The disclosure also relates to a method of predicting resistance to pesticides. The disclosure involves collection and use of genotypic sequence information and haplotyping of pests, in combination with remote sensing data and phenotyping data, to identify field level pesticide resistance.

### SUMMARY

An embodiment of the disclosure relates to a method of generating pesticide resistance information, such as a pesticide resistance map, of a target pest, the method comprising: obtaining genotypic information from a plurality of pest samples obtained from a plurality of field locations; generating one or more haplotypes based on the genotypic information, wherein a correlation exists between the haplotypes and resistance to at least one pesticide; correlating the haplotypes to the plurality of locations to generate a haplotype frequency information; and generating pesticide resistance information based on the haplotype frequency information.

In a further embodiment, the pest samples are obtained from the air, soil, water, plant part or a combination thereof.

In a further embodiment, the pest samples are spores.

In a further embodiment, the spores are spores of a species selected from the group consisting of Phakopsora pachyrizi and Septoria glycines.

In a further embodiment, the pest samples are from Phakopsora pachyrizi.

In a further embodiment, the pest samples are from Septoria glycines.

In a further embodiment, the obtaining step also comprises assaying soy plant leaf tissue.

In a further embodiment, generating the haplotypes comprises testing for alleles of the CYP51, SDHC, SDHB, SDHD, and CYTB genes.

In a further embodiment, the alleles of the CYP51, SDHC, SDHB, SDHD, and CYTB genes are correlated with resistance to at least one pesticide to develop the pesticide resistance factors.

In a further embodiment, the one or more haplotypes are associated with one or more pesticide resistance factors, and generating the pesticide resistance information comprises correlating the pesticide resistance factors with the haplotype frequency information.

In a further embodiment, correlating the pesticide resistance factors comprises generating a weighted sum of the pesticide resistance factors for at least two haplotypes, where the weight is based on the relative frequency of each haplotype.

In a further embodiment additionally comprises obtaining a plurality of spectral images of the plurality of field locations; and identifying a plurality of localized disease states based on the plurality of spectral images; and generating the pesticide resistance information additionally comprises correlating the plurality of localized disease states with the haplotype frequency information.

In a further embodiment, obtaining a plurality of spectral images of the plurality of field locations comprises monitoring an aerial vehicle (AV) as the AV flies along a flight path above the plurality of locations and as the AV performs capturing a plurality of images of the plurality of locations as the AV flies along the flight path; and transmitting the plurality of images to an image recipient.

In a further embodiment, the aerial vehicle is an unmanned aerial vehicle (UAV).

In a further embodiment, obtaining a plurality of spectral images of the plurality of field locations comprises obtaining a plurality of remote sensing-generated images of the field locations.

In a further embodiment, identifying a plurality of localized disease states comprises obtaining a plurality of vegetation index values based on the plurality of spectral images.

In a further embodiment, the vegetation index values are selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the plurality of field locations are soy fields.

In a further embodiment, the genotypic information is obtained at a location remote from the plurality of field locations.

In a further embodiment, the genotypic information is obtained within one week of obtaining the plurality of pest samples.

In a further embodiment, the genotypic information is obtained in the plurality of field locations.

In a further embodiment, obtaining genotypic information is conducted contemporaneously with collecting pest samples.

In a further embodiment, the genotypic information is obtained using a portable detection unit.

In a further embodiment, the correlation between the haplotypes and resistance to at least one pesticide is based on data from at least one previous season.

In a further embodiment, the correlation between the haplotypes and resistance to at least one pesticide is based on data from at least three previous seasons.

In a further embodiment, the correlation between the haplotypes and resistance to at least one pesticide is based on data from at least five previous seasons.

In a further embodiment, a recommended spray protocol is additionally generated for a particular location based on the pesticide resistance information.

In a further embodiment, generating the recommended spray protocol additionally comprises a recommended pesticide and a recommended application timing.

An embodiment of the present disclosure relates to a method of providing a real-time recommended pesticide application protocol based on identifying resistance to at least one pesticide in a location, comprising: collecting pest samples in the location; obtaining information from the pest samples; associating the information with pesticide resistance factors; and providing the real-time recommended pesticide application protocol based on the pesticide resistance factors.

In a further embodiment, the obtained information is additionally compared to historic information previously obtained in the location; and changes in pesticide resistance are identified based on the results of the comparison; and the real-time recommended pesticide application protocol is provided based on the pesticide resistance factors and the changes in pesticide resistance.

In a further embodiment, the historic information was obtained during at least one previous growing season.

In a further embodiment, the historic information was obtained during at least three previous growing seasons.

In a further embodiment, the historic information was obtained during at least five previous growing seasons.

In a further embodiment, at least one spectral image of the location is additionally obtained; at least one localized disease state is identified based on the spectral image; and the real-time recommended pesticide application protocol is additionally provided based on the at least one localized disease state.

In a further embodiment, obtaining at least one spectral image of the location comprises monitoring an aerial vehicle (AV) as the AV flies along a flight path above the location and as the AV performs: capturing at least one image of the location as the AV flies along the flight path; and transmitting the image to an image recipient.

In a further embodiment, the aerial vehicle is an unmanned aerial vehicle (UAV).

In a further embodiment, the image recipient also obtains the information from the pest samples.

In a further embodiment, obtaining at least one spectral image of the location comprises obtaining at least one satellite-generated image of the location.

In a further embodiment, identifying at least one localized disease state comprises obtaining at least one vegetation index value based on the spectral image.

In a further embodiment, the vegetation index value is selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the information is genotypic sequence information.

In a further embodiment, the genotypic sequence information is used to generate at least one haplotype.

In a further embodiment, the information is metabolic information.

In a further embodiment, the information is a genetic marker.

In a further embodiment, the choice of the assay for the genetic marker is based on historic pesticide resistance information.

In a further embodiment, the information is protein expression information.

In a further embodiment, the information is genetic transcript information.

In a further embodiment, the recommended pesticide application protocol relates to a pesticide application dose.

In a further embodiment, the recommended pesticide application protocol relates to timing of pesticide application.

In a further embodiment, the pest samples are obtained from the air, soil, water, plant part or a combination thereof.

In a further embodiment, the pest samples are spores.

In a further embodiment, the spores are spores of Phakopsora pachyrizi.

In a further embodiment, the location is a soy field.

In a further embodiment, the information is obtained at a location remote from the plurality of field locations.

In a further embodiment, the information is obtained within one week of obtaining the plurality of pest samples.

In a further embodiment, the information is obtained in the plurality of field locations.

In a further embodiment, the information is obtained contemporaneously with collecting pest samples.

In a further embodiment, the information is obtained using a portable detection unit.

Another embodiment of the present disclosure relates to a method of prescribing a targeted application of a crop protection agent to reduce a plant disease in a grower's field, the method comprising: obtaining sequence information of one or more pests from the grower's field, wherein the field comprises a population of plants suspected of exhibiting the plant disease; accessing a plurality of images of one or more of the plants in the plant population to enable identification of the suspected plant disease; analyzing the sequence information obtained from the pests and determining pesticide resistance characteristics of the pest that causes the plant disease; and providing a prescription of the crop protection agent that is effective to control the pests, wherein the pests do not exhibit substantial resistance to the crop protection agent.

In a further embodiment, accessing a plurality of images comprises monitoring an aerial vehicle (AV) as the AV flies along a flight path above the grower's field and as the AV performs: capturing a plurality of images of the location as the AV flies along the flight path; and transmitting the images to an image recipient.

In a further embodiment, the image recipient also obtains the sequence information of one or more pests.

In a further embodiment, accessing a plurality of images comprises obtaining a plurality of satellite-generated images of the grower's field.

In a further embodiment, identification of the suspected plant disease comprises obtaining at least one vegetation index value based on the plurality of images.

In a further embodiment, the vegetation index value is selected from the group consisting of a normalized difference vegetation index (NDVI), a land surface water index (LSWI), a temperature-vegetation dryness index (TVDI), a soil adjusted vegetation index (SAVI), and a water deficit index (WDI).

In a further embodiment, the sequence information is used to generate at least one haplotype.

In a further embodiment, the prescription of the crop protection agent relates to a crop protection agent application dose.

In a further embodiment, the prescription of the crop protection agent relates to timing of crop protection agent application.

In a further embodiment, the one or more pests are obtained from the air, soil, water, plant part or a combination thereof.

In a further embodiment, the one or more pests are spores.

In a further embodiment, prescribing a targeted application of a crop protection agent comprises generating a weighted sum of resistance factors.

In a further embodiment, the grower's field is a soy field.

In a further embodiment, the sequence information is obtained at a location remote from the grower's field.

In a further embodiment, the sequence information is obtained within one week of obtaining a sample of one or more pests from the grower's field.

In a further embodiment, the sequence information is obtained in the grower's field.

In a further embodiment, the information is obtained using a portable detection unit.

In a further embodiment, the crop protection agent is a pesticide.

In a further embodiment, the pesticide belongs to a class of fungicides selected from the group consisting of aliphatic nitrogen fungicides, amide fungicides, antibiotic fungicides, aromatic fungicides, arsenical fungicides, aryl phenyl ketone fungicides, benzimidazole fungicides, benzimidazole precursor fungicides, botanical fungicides, bridged diphenyl fungicides, carbamate fungicides, conazole fungicides, copper fungicides, cyanoacrylate fungicides, dicarboximide fungicides, dinitrophenol fungicides, dithiocarbamate fungicides, dithiolane fungicides, fumigant fungicides, hydrazide fungicides, imidazole fungicides, inorganic fungicides, mercury fungicides, morpholine fungicides, organophosphorus fungicides, organotin fungicides, oxathiin fungicides, oxazole fungicides, polysulfide fungicides, pyrazole fungicides, pyridazine fungicides, pyridine fungicides, pyrimidine fungicides, pyrrole fungicides, quaternary ammonium fungicides, quinoline fungicides, quinone fungicides, quinoxaline fungicides, tetrazole fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, zinc fungicides, and unclassified fungicides.

In a further embodiment, the pesticide is an aliphatic nitrogen fungicide selected from the group consisting of butylamine, cymoxanil, dodicin, dodine, guazatine, iminoctadine, and xinjunan.

In a further embodiment, the pesticide belongs to a subclass of amide fungicides selected from the group consisting of acylamino acid fungicides, anilide fungicides, benzamide fungicides, furamide fungicides, phenylsulfamide fungicides, picolinamide fungicides, pyrazolecarboxamide fungicides, sulfonamide fungicides, and valinamide fungicides.

In a further embodiment, the pesticide is an amide fungicide selected from the group consisting of carpropamid, chloraniformethan, cyflufenamid, diclocymet, diclocymet, dimoxystrobin, fenaminstrobin, fenoxanil, flumetover, isofetamid, mandestrobin, mandipropamid, metominostrobin, orysastrobin, prochloraz, quinazamid, silthiofam, triforine, and trimorphamide.

In a further embodiment, the pesticide is an acylamino acid fungicide selected from the group consisting of benalaxyl, benalaxyl-M, furalaxyl, metalaxyl, metalaxyl-M, pefurazoate, and valifenalate.

In a further embodiment, the pesticide is an anilide fungicide selected from the group consisting of benalaxyl, benalaxyl-M, bixafen, boscalid, carboxin, fenhexamid, flubeneteram, fluxapyroxad, isotianil, metalaxyl, metalaxyl-M, metsulfovax, ofurace, oxadixyl, oxycarboxin, penflufen, pyracarbolid, pyraziflumid, sedaxane, thifluzamide, tiadinil, and vangard.

In a further embodiment, the pesticide belongs to a further subclass of anilide fungicides selected from the group consisting of benzanilide fungicides, furanilide fungicides, and sulfonanilide fungicides.

In a further embodiment, the pesticide is a benzanilide fungicide selected from the group consisting of benodanil, flutolanil, mebenil, mepronil, salicylanilide, and tecloftalam.

In a further embodiment, the pesticide is a furanilide fungicide selected from the group consisting of fenfuram, furalaxyl, furcarbanil, and methfuroxam.

In a further embodiment, the pesticide is a sulfonanilide fungicide selected from the group consisting of flusulfamide and tolnifanide.

In a further embodiment, the pesticide is a benzamide fungicide selected from the group consisting of benzohydroxamic acid, fluopicolide, fluopimomide, fluopyram, tioxymid, trichlamide, zarilamid, and zoxamide.

In a further embodiment, the pesticide is a furamide fungicide selected from the group consisting of cyclafuramid and furmecyclox.

In a further embodiment, the pesticide is a phenylsulfamide fungicide selected from the group consisting of dichlofluanid and tolylfluanid.

In a further embodiment, the pesticide is a picolinamide fungicide selected from the group consisting of fenpicoxamid and florylpicoxamid.

In a further embodiment, the pesticide comprises fenpicoxamid.

In a further embodiment, the pesticide is a pyrazolecarboxamide fungicide selected from the group consisting of benzovindiflupyr, bixafen, flubeneteram, fluindapyr, fluxapyroxad, furametpyr, inpyrfluxam, isopyrazam, penflufen, penthiopyrad, pydiflumetofen, pyrapropoyne, and sedaxane.

In a further embodiment, the pesticide is a sulfonamide fungicide selected from the group consisting of amisulbrom, cyazofamid, and dimefluazole.

In a further embodiment, the pesticide is a valinamide fungicide selected from the group consisting of benthiavalicarb and iprovalicarb.

In a further embodiment, the pesticide is an antibiotic fungicide selected from the group consisting of aureofungin, blasticidin-S, cycloheximide, fenpicoxamid, griseofulvin, kasugamycin, moroxydine, natamycin, ningnanmycin, polyoxins, polyoxorim, streptomycin, and validamycin.

In a further embodiment, the pesticide belongs to the subclass of antibiotic fungicides comprising strobilurin fungicides.

In a further embodiment, the pesticide is a strobilurin fungicide selected from the group consisting of fluoxastrobin, mandestrobin, and pyribencarb.

In a further embodiment, the pesticide belongs to a further subclass of strobilurin fungicides selected from the group consisting of methoxyacrylate strobilurin fungicides, methoxycarbanilate strobilurin fungicides, methoxyiminoacetamide strobilurin fungicides, and methoxyiminoacetate strobilurin fungicides.

In a further embodiment, the pesticide is a methoxyacrylate strobilurin fungicide selected from the group consisting of azoxystrobin, bifujunzhi, coumoxystrobin, enoxastrobin, flufenoxystrobin, jiaxiangjunzhi, picoxystrobin, and pyraoxystrobin.

In a further embodiment, the pesticide is a methoxycarbanilate strobilurin fungicide selected from the group consisting of pyraclostrobin, pyrametostrobin, and triclopyricarb.

In a further embodiment, the pesticide is a methoxyiminoacetamide strobilurin fungicide selected from the group consisting of dimoxystrobin, fenaminstrobin, metominostrobin, and orysastrobin.

In a further embodiment, the pesticide is a methoxyiminoacetate strobilurin fungicide selected from the group consisting of kresoxim-methyl and trifloxystrobin.

In a further embodiment, the pesticide is an aromatic fungicide selected from the group consisting of biphenyl chlorodinitronaphthalenes, chloroneb, chlorothalonil, cresol, dicloran, fenjuntong, hexachlorobenzene, pentachlorophenol, quintozene, sodium pentachlorophenate, tecnazene, thiocyanatodinitrobenzenes, and trichlorotrinitrobenzenes.

In a further embodiment, the pesticide is an arsenical fungicide selected from the group consisting of asomate and urbacide.

In a further embodiment, the pesticide is an aryl phenyl ketone fungicide selected from the group consisting of metrafenone and pyriofenone.

In a further embodiment, the pesticide is a benzimidazole fungicide selected from the group consisting of albendazole, benomyl, carbendazim, chlorfenazole, cypendazole, debacarb, dimefluazole, fuberidazole, mecarbinzid, rabenzazole, and thiabendazole.

In a further embodiment, the pesticide is a benzimidazole precursor fungicide selected from the group consisting of furophanate, thiophanate, and thiophanate-methyl.

In a further embodiment, the pesticide is a benzothiazole fungicide selected from the group consisting of bentaluron, benthiavalicarb, benthiazole, chlobenthiazone, dichlobentiazox, and probenazole.

In a further embodiment, the pesticide is a botanical fungicide selected from the group consisting of allicin, berberine, carvacrol, carvone, osthol, sanguinarine, and santonin.

In a further embodiment, the pesticide is a bridged diphenyl fungicide selected from the group consisting of bithionol, dichlorophen, diphenylamine, hexachlorophene, and parinol.

In a further embodiment, the pesticide is a carbamate fungicide selected from the group consisting of benthiavalicarb, furophanate, iodocarb, iprovalicarb, picarbutrazox, propamocarb, pyribencarb, thiophanate, thiophanate-methyl, and tolprocarb.

In a further embodiment, the pesticide belongs to a subclass of carbamate fungicides selected from the group consisting of benzimidazolylcarbamate fungicides and carbanilate fungicides.

In a further embodiment, the pesticide is a benzimidazolylcarbamate fungicide selected from the group consisting of albendazole, benomyl, carbendazim, cypendazole, debacarb, and mecarbinzid.

In a further embodiment, the pesticide is a carbanilate fungicide selected from the group consisting of diethofencarb, pyraclostrobin, pyrametostrobin, and triclopyricarb.

In a further embodiment, the pesticide belongs to a subclass of conazole fungicides selected from the group consisting of imidazoles and triazoles.

In a further embodiment, the pesticide is an imidazole fungicide selected from the group consisting of climbazole, clotrimazole, imazalil, oxpoconazole, prochloraz, and triflumizole.

In a further embodiment, the pesticide is a triazole fungicide selected from the group consisting of azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, ipfentrifluconazole, mefentrifluconazole, metconazole, myclobutanil, penconazole, propiconazole, prothioconazole, quinconazole, simeconazole, tebuconazole, tetraconazole, triadimefon, triadimenol, triticonazole, uniconazole, and uniconazole-P.

In a further embodiment, the pesticide is a carbamate fungicide selected from the group consisting of acypetacs-copper, basic copper carbonate, basic copper sulfate, Bordeaux mixture, Burgundy mixture, Cheshunt mixture, copper acetate, copper hydroxide, copper naphthenate, copper oleate, copper oxychloride, copper silicate, copper sulfate, copper zinc chromate, cufraneb, cuprobam, cuprous oxide, mancopper, oxine-copper, saisentong, and thiodiazole-copper.

In a further embodiment, the pesticide is a cyanoacrylate fungicide selected from the group consisting of benzamacril and phenamacril.

In a further embodiment, the pesticide is a dicarboximide fungicide selected from the group consisting of famoxadone and fluoroimide.

In a further embodiment, the pesticide belongs to a subclass of dicarboximide fungicides selected from the group consisting of dichlorophenyl dicarboximide fungicides and phthalimide fungicides.

In a further embodiment, the pesticide is a dichlorophenyl dicarboximide fungicide selected from the group consisting of chlozolinate, dichlozoline, iprodione, isovaledione, myclozolin, procymidone, and vinclozolin.

In a further embodiment, the pesticide is a phthalimide fungicide selected from the group consisting of captafol, captan, ditalimfos, folpet, and thiochlorfenphim.

In a further embodiment, the pesticide is a dinitrophenol fungicide selected from the group consisting of binapacryl, dinobuton, dinocap, dinocap-4, dinocap-6, meptyldinocap, dinocton, dinopenton, dinosulfon, dinoterbon, and DNOC.

In a further embodiment, the pesticide is a dithiocarbamate fungicide selected from the group consisting of amobam, asomate, azithiram, carbamorph, cufraneb, cuprobam, disulfiram, ferbam, metam, nabam, tecoram, thiram, urbacide, and ziram.

In a further embodiment, the pesticide belongs to a subclass of dithiocarbamate fungicides selected from the group consisting of cyclic dithiocarbamate fungicides and polymeric dithiocarbamate fungicides.

In a further embodiment, the pesticide is a cyclic dithiocarbamate fungicide selected from the group consisting of dazomet, etem, and milneb.

In a further embodiment, the pesticide is a polymeric dithiocarbamate fungicide selected from the group consisting of mancopper, mancozeb, maneb, metiram, polycarbamate, propineb, and zineb.

In a further embodiment, the pesticide is a dithiolane fungicide selected from the group consisting of isoprothiolane and saijunmao.

In a further embodiment, the pesticide is a fumigant fungicide selected from the group consisting of allyl isothiocyanate, carbon disulfide, cyanogen, dimethyl disulfide, methyl bromide, methyl iodide, methyl isothiocyanate, and sodium tetrathiocarbonate.

In a further embodiment, the pesticide is a hydrazide fungicide selected from the group consisting of benquinox, chloroinconazide, and saijunmao.

In a further embodiment, the pesticide is an imidazole fungicide selected from the group consisting of cyazofamid, fenamidone, fenapanil, glyodin, iprodione, isovaledione, pefurazoate, and triazoxide.

In a further embodiment, the pesticide is a member of the conazole fungicides subclass of the imidazole fungicides selected from the group consisting of climbazole, clotrimazole, imazalil, oxpoconazole, prochloraz, and triflumizole.

In a further embodiment, the pesticide is an inorganic fungicide selected from the group consisting of potassium azide, potassium thiocyanate, sodium azide, and sulfur.

In a further embodiment, the pesticide belongs to a subclass of mercury fungicides selected from the group consisting of inorganic mercury fungicides and organomercury fungicides.

In a further embodiment, the pesticide is an inorganic mercury fungicide selected from the group consisting of mercuric chloride, mercuric oxide, and mercurous chloride.

In a further embodiment, the pesticide is an organomercury fungicide selected from the group consisting of (3-ethoxypropyl) mercury bromide, ethylmercury acetate, ethylmercury bromide, ethylmercury chloride, ethylmercury 2,3-dihydroxypropyl mercaptide, ethylmercury phosphate, N−(ethylmercury)-p-toluenesulfonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriureaphenylmercury acetate, phenylmercury chloride, phenylmercury derivative of pyrocatechol, phenylmercury nitrate, phenylmercury salicylate, thiomersal, and tolylmercury acetate.

In a further embodiment, the pesticide is a morpholine fungicide selected from the group consisting of aldimorph, benzamorf, carbamorph, dimethomorph, dodemorph, fenpropimorph, flumorph, tridemorph, and trimorphamide.

In a further embodiment, the pesticide is an organophosphorus fungicide selected from the group consisting of ampropylfos, ditalimfos, EBP, edifenphos, fosetyl, hexylthiofos, inezin, iprobenfos, izopamfos, kejunlin, phosdiphen, pyrazophos, tolclofos-methyl, and triamiphos.

In a further embodiment, the pesticide is an organotin fungicide selected from the group consisting of decafentin, fentin, and tributyltin oxide.

In a further embodiment, the pesticide is an oxathiin fungicide selected from the group consisting of carboxin and oxycarboxin.

In a further embodiment, the pesticide is an oxazole fungicide selected from the group consisting of chlozolinate, dichlozoline, drazoxolon, famoxadone, fluoxapiprolin, hymexazol, metazoxolon, myclozolin, oxadixyl, oxathiapiprolin, pyrisoxazole, and vinclozolin.

In a further embodiment, the pesticide is a polysulfide fungicide selected from the group consisting of barium polysulfide, calcium polysulfide, potassium polysulfide, and sodium polysulfide.

In a further embodiment, the pesticide is a pyrazole fungicide selected from the group consisting of oxathiapiprolin, fluoxapiprolin, and rabenzazole.

In a further embodiment, the pesticide belongs to a subclass of pyrazole fungicides selected from the group consisting of phenylpyrazole fungicides and pyrazolecarboxamide fungicides.

In a further embodiment, the pesticide is a phenylpyrazole fungicide selected from the group consisting of fenpyrazamine, metyltetraprole, pyraclostrobin, pyrametostrobin, and pyraoxystrobin.

In a further embodiment, the pesticide is a pyrazolecarboxamide fungicide selected from the group consisting of benzovindiflupyr, bixafen, flubeneteram, fluindapyr, fluxapyroxad, furametpyr, inpyrfluxam, isoflucypram, isopyrazam, penflufen, penthiopyrad, pydiflumetofen, and sedaxane.

In a further embodiment, the pesticide is the pyridazine fungicide pyridachlometyl.

In a further embodiment, the pesticide is a pyridine fungicide selected from the group consisting of aminopyrifen, boscalid, buthiobate, dipyrithione, fluazinam, fluopicolide, fluopyram, parinol, picarbutrazox, pyribencarb, pyridinitril, pyrifenox, pyrisoxazole, pyroxychlor, pyroxyfur, and triclopyricarb.

In a further embodiment, the pesticide is a pyrimidine fungicide selected from the group consisting of bupirimate, diflumetorim, dimethirimol, ethirimol, fenarimol, ferimzone, nuarimol, and triarimol.

In a further embodiment, the pesticide belongs to the anilinopyrimidine fungicide subclass of the pyrazole fungicides.

In a further embodiment, the pesticide is an anilinopyrimidine fungicide selected from the group consisting of cyprodinil, mepanipyrim, and pyrimethanil.

In a further embodiment, the pesticide is a pyrrole fungicide selected from the group consisting of dimetachlone, fenpiclonil, fludioxonil, and fluoroimide.

In a further embodiment, the pesticide is a quaternary ammonium fungicide selected from the group consisting of berberine and sanguinarine.

In a further embodiment, the pesticide is a quinoline fungicide selected from the group consisting of ethoxyquin, halacrinate, 8-hydroxyquinoline sulfate, ipflufenoquin, quinacetol, quinofumelin, quinoxyfen, and tebufloquin.

In a further embodiment, the pesticide is a quinone fungicide selected from the group consisting of chloranil, dichlone, and dithianon.

In a further embodiment, the pesticide is a quinoxaline fungicide selected from the group consisting of chinomethionat, chlorquinox, and thioquinox.

In a further embodiment, the pesticide is a tetrazole fungicide selected from the group consisting of metyltetraprole and picarbutrazox.

In a further embodiment, the pesticide is a thiadiazole fungicide selected from the group consisting of etridiazole, saisentong, thiodiazole-copper, and zinc thiazole.

In a further embodiment, the pesticide is a thiazole fungicide selected from the group consisting of dichlobentiazox, ethaboxam, fluoxapiprolin, isotianil, metsulfovax, octhilinone, oxathiapiprolin, thiabendazole, and thifluzamide.

In a further embodiment, the pesticide is a thiazolidine fungicide selected from the group consisting of flutianil and thiadifluor.

In a further embodiment, the pesticide is a thiocarbamate fungicide selected from the group consisting of methasulfocarb and prothiocarb.

In a further embodiment, the pesticide is a thiophene fungicide selected from the group consisting of ethaboxam, isofetamid, penthiopyrad, silthiofam, and thicyofen.

In a further embodiment, the pesticide is the triazine fungicide anilazine.

In a further embodiment, the pesticide is a triazole fungicide selected from the group consisting of amisulbrom, bitertanol, fluotrimazole, and triazbutil.

In a further embodiment, the pesticide belongs to the conazole fungicide subclass of the triazole fungicides.

In a further embodiment, the pesticide is a conazole fungicide selected from the group consisting of azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, huanjunzuo, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, and uniconazole-P.

In a further embodiment, the pesticide is the triazolopyrimidine fungicide ametoctradin.

In a further embodiment, the pesticide is a urea fungicide selected from the group consisting of bentaluron, pencycuron, and quinazamid.

In a further embodiment, the pesticide is a zinc fungicide selected from the group consisting of acypetacs-zinc, copper zinc chromate, cufraneb, mancozeb, metiram, polycarbamate, polyoxorim-zinc, propineb, zinc naphthenate, zinc thiazole, zinc trichlorophenate, zineb, and ziram.

In a further embodiment, the pesticide belongs to the unclassified fungicide class and is selected from the group consisting of acibenzolar, acypetacs, allyl alcohol, benzalkonium chloride, bethoxazin, bromothalonil, chitosan, chloropicrin, DBCP, dehydroacetic acid, diclomezine, diethyl pyrocarbonate, dipymetrone, ethylicin, fenaminosulf, fenitropan, fenpropidin, formadehyde, furfural, hexachlorobutadiene, nitrostyrene, nitrothal-isopropyl, OCH, oxyfenthiin, pentachlorophenyl laurate, 2-phenylphenol, phthalide, piperalin, propamidine, proquinazid, pyroquilon, sodium o-phenylphenoxide, spiroxamine, sultropen, and tricyclazole.

A further embodiment provides a method of generating pesticide resistance information of a target pest, the method comprising: obtaining genotypic information from a plurality of pest samples obtained from a plurality of field locations; generating the frequency of one or more genotypes based on the genotypic information, wherein a correlation exists between the genotypes and resistance to at least one pesticide, wherein the correlation is quantified as at least one pesticide resistance factor to at least one pesticide; correlating the genotypes to the plurality of locations to generate genotype frequency information; and generating pesticide resistance information based on the genotype frequency information and the pesticide resistance factor of that genotype.

In a further embodiment, the method further comprises: identifying candidate pesticides for use in a pesticide application protocol based on the pesticide resistance information; generating a provisional pesticide application protocol for a field location using the pesticide resistance information;
selecting, based on the pesticide resistance information, a plurality of validating assays for the provisional pesticide application protocol; and performing the assays to generate a validated pesticide application protocol.

In a further embodiment of the method, pre-existing data for the field location are employed in generating the provisional pesticide application protocol.

In a further embodiment of the method, the assays are performed in the field location.

In a further embodiment of the method, the fungal materials are from a species selected from the group consisting of Phakopsora pachyrizi and Septoria glycines.

In a further embodiment of the method, the pest samples are from Phakopsora pachyrizi.

In a further embodiment of the method, the pest samples are from Septoria glycines.

In a further embodiment of the method, the genotypes are generated by testing for alleles of genes involved in resistance to pesticides.

In a further embodiment of the method, the alleles are alleles of the CYP51, SDHC, SDHB, SDHD, CYTB, OSBP and multi-drug resistance genes.

In a further embodiment of the method, the alleles of the CYP51, SDHC, SDHB, SDHD, CYTB, OSBP and multi-drug resistance genes are correlated with resistance to at least one pesticide to develop the pesticide resistance factors.

A further embodiment provides a method of developing a recommended pesticide application protocol, the method comprising: based on pesticide resistance information, determining recommended assays to identify a frequency of a plurality of genotypes in a plurality of pest samples obtained from a field location, wherein a correlation exists between the genotypes and resistance to at least one pesticide, wherein the resistance is quantified as a plurality of resistance factors; performing the recommended assays; calculating a weighted average of the plurality of resistance factors based on the frequency of different genotypes to obtain an estimated location-specific resistance factor for the pesticide; and developing the recommended pesticide application protocol based on the estimated location-specific resistance factor.

A further embodiment provides a method of providing a real-time recommended pesticide application protocol based on identifying resistance of a pest to at least one pesticide in a field location, comprising: collecting pest samples in the field location; obtaining abundance and frequency information of specific genotypes from the pest samples; associating one or more genotypes with information on pesticide resistance factors; calculating a location specific pesticide resistance factor for at least one pesticide based on the frequency of the genotypes and their associated pesticide resistance factor, and providing the real-time recommended pesticide application protocol based on the location-specific pesticide resistance factors.

A further embodiment of the method additionally comprises: comparing the obtained genotypic information to historic or pre-existing genotypic information for the location; and identifying changes in pesticide resistance based on the results of the comparison; wherein providing the real-time recommended pesticide application protocol is based on the pesticide resistance factors and the changes in pesticide resistance.

A further embodiment provides a method of prescribing a targeted application of a crop protection agent to reduce a plant disease in a field location, the method comprising: obtaining genotypic information of one or more pests from pest samples obtained from the field location, wherein the field location comprises a population of plants suspected of exhibiting the plant disease; accessing a plurality of images of one or more of the plants in the plant population to enable identification of the suspected plant disease; analyzing the genotypic information obtained from the pests and determining resistance characteristics of the pest that causes the plant disease to obtain at least one resistance factor; and providing a prescription of the crop protection agent that is effective to control the pests, wherein the pests do not exhibit substantial resistance to the crop protection agent.

In a further embodiment, the genotypes comprise Y131H in the CYP51 gene.

### DETAILED DESCRIPTION

The present disclosure relates to a method of assessing the state of plant disease in a field location by obtaining information related to the pest responsible for the plant disease and its susceptibility to one or more pesticides. The information may be directly obtained in the field location, or samples of the pest may be obtained and transported to a remote location where the information is obtained. The information may be genotypic information, may relate to the metabolic state of the pest, or may be developed as a result of biochemical analysis of the pest, or analysis of the transcriptome or proteome of the pest. In a preferred embodiment, the information relates to haplotypes based on genotypic information related to the pest.

The present disclosure also relates to the combination of the specific information related to the pest's susceptibility to one or more pesticides with remote sensing information obtained via an aerial sensor or satellite imagery. Such imagery may be used to identify areas of the field location either currently subject to the plant disease or predisposed to develop the plant disease. The imagery may be combined in real time with the information related to the pest's susceptibility to one or more pesticides, or may be used to identify areas of the field location in which the information related to the pest's susceptibility is to be obtained.

The present disclosure also relates to the use of information related to the pest responsible for the plant disease and its susceptibility to one or more pesticides to generate a recommended treatment protocol for the field location in which the information is obtained. In a preferred embodiment, the treatment protocol is generated in real time based on information obtained in the field location. The treatment protocol may also be based in part on remote sensing information obtained via an aerial camera or satellite imagery. The recommended treatment protocol may relate to the recommendation or one or a combination of pesticides to be employed to combat the pest responsible for the plant disease, or may related to a recommended timing of application, or application dosages, or any combination thereof.

The present disclosure also relates to the compilation of information related to the pest responsible for the plant disease over time, and the use of that historical information to track changes in susceptibility of the pest, past treatment protocols and responses, and to generate current recommended treatment protocols.

### Acquisition of Remote Sensing Data

In agricultural applications, remote sensing involves obtaining, from a distance, information about agricultural systems, especially spatial or temporal variations in those systems. Such remote sensing generally involves sensors mounted on a satellite or an airborne vehicle. The sensors generally measure electromagnetic radiation, which can be either reflected or emitted by the system. The information obtained can relate, among other characteristics, to chemical, morphological, or biological properties of the plants.

The remote sensing data obtained can be used to calculate traits of the plants in the agricultural system. These traits can be used to determine the existence of and state of progression of disease-borne plant illnesses. Such traits can be determined from the difference in spectral data detected between healthy plants and infected ones, or between known data from plants at a specific state of disease progression and currently-obtained spectral data from the field. Sec Bravo et al., Biosyst. Eng. 84, 137-145 (2003); Mahlein, Plant Disease 100, 241-251 (2015). The data obtained are generally more useful with increasing spatial resolution, which permits assessment of, e.g, plant shape or spatial pattern. See Lopez-Granados, Weed Research 51, 1-11 (2010). Particularly in cases or rapidly-developing plant diseases, timing of data acquisition can also be important. In such cases, sensing data from maanned or unmanned aerial vehicles (e.g. drones) may be preferred, as such vehicles offer control of the timing of access and ability to repeat data acquisition as desired, in comparison to satellite-based data, which generally offer data acquisition windows that may be more separated in time and little to no ability to control the timing of such windows.

However, increasingly even satellite-based data (e.g. Sentinel-2 imagery) offers a useful level of resolution, in the meter to decameter range, and useful data acquisition windows of e. g., 5 days. Satellite platforms currently useful for agricultural applications include the American Landsat satellites (eight satellites taking spectral data from the Earth each 16 to 18 days), the European Sentinel 2 satellite system (providing multispectral data at 10 m pixel resolution for NDVI imagery, soil, and water cover every ten days), the RapidEye constellation (five satellites providing multispectral RGB imagery, as well as red-edge and NIR bands at 5 m resolution), the GeoEye-1 system (capturing multispectral RGB data and NIR data at a 1.84 m resolution), and the WorldView-3 (collecting multispectral data from the RGB bands including the red-edge, two NIR bands, and 8 SWIR bands with a resolution of 1.24 m at nadir). The development of further satellite platforms useful for such application is to be expected.

In a preferred embodiment, the Doves platform of Earth-imaging satellites established by Planet Labs, Inc. (www.planet.com) is employed to obtain the remote imaging data. This platform provides a daily stream of imagery at the 3-5 meter resolution scale.

The measurement of traits of plants in the field begins with the retrieval of primary sensing data. This data may be obtained through the use of various types of sensory apparatus, such as multi- or hyperspectral sensors, fluorescence sensors, photogrammetrical sensors, Light Detection and Ranging (LIDAR) scanners, and the like. From this data, primary variables can be developed. These include, for example, plant density or plant organ counting, green area index (GAI) or leaf area index (LAI), leaf biochemical content, leaf orientation, crop height, fraction of absorbed photosynthetically active radiation (fAPAR), and water content. Such primary variables can be developed by regression analysis; by mechanistic modeling such as iterative optimization techniques, look-up-table approaches, machine learning, stereovision/photogrammetric methods, LIDAR signal processing of laser pulse backscattering, or interferometric SAR; or by methods relying on classification and segmentation techniques, such as object-based image analysis. Furthermore, these may be reported in terms of various indices, such as, for example, a normalized difference vegetation index (NDVI), a land surface water index (LSWI), a temperature-vegetation dryness index (TVDI), a soil adjusted vegetation index (SAVI), or a water deficit index (WDI). These primary variables may then be used to develop secondary variables that are only indirectly linked to the remote sensing data. These secondary variables may relate to, for example, estimated crop yield, canopy radiation use efficiency, crop coefficient, and crop nitrogen content.

An aerial vehicle (AV) or an unmanned aerial vehicle (AV) may be used to acquire the remote sensing data. The unmanned aerial vehicle may be either fixed-wing or, preferably, capable of hovering. The may include a global positioning system (GPS) that provides the location of the AV or UAV. The UAV may either be automatically controlled or may be remotely piloted by a human operator. Preferably, the AV or UAV contains sonar or other form of height sensor to accurately determine elevation during operation. The AV or UAV may also include sensors to determine the speed and attitude of the UAV. The AV or UAV may incorporate an autopilot function. Preferably, the AV or UAV has a communication system to communicate with a base station during operation.

The UAV preferably includes a sensor package mounted thereto for obtaining sensory data during operation. The sensor package preferably allows rotation about one or more axes. In some cases, it may be directed via actuators. The sensor package may include, for example, a camera for obtaining RGB imagery, a thermal imaging camera, or a near infrared camera for obtaining NDVI images. The sensor package may be controlled by on-board elements such as a CPU, which may, for example, control the orientation and operation of the sensor package, dependent on ambient conditions.

Sensors that rely on light disruption caused by certain pests such as insects to identify are also used in conjunction with genotypic data generated herein. Such pseudo acoustic sensors generate remote sensing data that are then coupled with one or more location specific genotypic information to develop resistance information to one or more pesticides. In an embodiment, such a sensor tracks the movement of an insect's wing beats with, for example a laser and a phototransistor array. This data based on light disruption is then converted to a sound file, which are then processed with algorithms capable of identifying the pest associated with the sound signature or wing-beat profile.

A computer may serve as the interface for the human operator. The computer may be in the form of a mobile device such as a mobile phone or tablet. In such cases, preferably the human operator interacts with the mobile device via a graphical interface. Via this interface, the operator can control, for example, the movement of the UAV or orientation and activation of the sensor package or any individual sensor. Preferably, the graphical interface will permit the operator to access previously obtained data, and information developed based on that data, particularly information indicating the state of plant health or disease infestation.

### Acquisition of Information Indicating Pest Susceptibility to Disease Treatment

Preferably, the disclosed methods include obtaining information relating to susceptibility of the pest responsible for the disease to various treatments for the disease. This information can be of any type useful to indicate such susceptibility. Exemplary type of information include genotypic information such as haplotypes, biochemical information, information relating to pest metabolism, transcriptional information, or information relating to protein translation.

This information may be acquired in any manner customary in the art. For example, genotypic information may be acquired by isolating and sequencing DNA of the pest organism. Such DNA may be acquired from spore samples. In alternate embodiments, DNA may be obtained from the air. In preferred embodiments, test kits may be prepared for previously-identified haplotypes indicating pest susceptibility to disease treatments such as application of a pesticide. These kits may comprise a set of nucleic acid probes, each comprising a nucleotide sequence that is specifically hybridizable to a nucleotide sequence indicating a genetic variation linked to susceptibility to a pesticide. Preferably, these kits may be employed in a field setting to obtain genotyping information that may be employed in real-time.

In another embodiment, the information relates to proteomics or expression of genes to produce a particular protein or proteins in the pests. The expression of either single proteins, or multiple differentially expressed proteins, may be used as an indicator of susceptibility to a particular treatment for a pest-borne disease. Such analyses have been used, for example, to assess risk for insecticide resistance in certain populations (see, e.g., Shin and Smartt, J. Vector Ecology 41:1, 63-71 (2015) (expression of particular esterase alleles as markers for naled resistance in mosquitoes). Such analysis preferably takes into account relevant environmental considerations such as nitrogen and water sufficiency.

In some cases, the information may relate to the expression of pest genes at the transcriptional level, in particular genes related to detoxification. Such genes have been identified in certain pests, for example the Asian citrus psyllid. See Tian et al., Scientific Rep. 8, no. 12587 (2018) (identifying pest resistance to imidacloprid as mainly related to increased expression of the detoxifying genes CYP4C68 and CYP4G70).

### Machine Learning, Deep Learning Based Artificial Intelligence Computing Systems for Disease Management

In an embodiment, one or more of the variables described herein for example, genotypic information, phenotypic images, and/or disease management practices can be fed into a machine learning or deep learning algorithm. For example, a neural network architecture for computing one or more predicted resistance values from one or more inputs. The neural networks are configured to synthesize or learn from a plurality of inputs to produce an output—for example, one or more inputs to disease resistance information can be modeled using machine learning approaches involving Bayesian algorithms. One or more variables in the algorithms can have weights that are applied to each equation and optimized as the neural network is trained. Based on the amount of training information, the deep learning models or networks get better at producing more helpful outputs.

Individual machine learning networks (e.g., artificial neural networks-ANN; Convolutional Neural Networks (CNN) s) are described herein at general terms based on inputs, outputs, and type of neural network. Based on the various inputs, such as for example, genetic haplotype information and field effects realized from one or more disease management practices, one of ordinary skill in the art given data on the inputs, outputs, and type of machine or deep learning modules would be able to construct working embodiments.

In an embodiment, deep neural network includes a plurality of input factors that may be used to train resistance information-based management practices. These factors include for example, pest resistance histories, QTLs, SNPs, haplotypes, yield, environmental classifications, crop protection input, soil conditions, and other agronomic or breeding components.

Training data generally refers to datasets that are used to train specific deep learning networks, such as for example, neural network. Each dataset may correspond to set of actual yield values or pest control values and the underlying management practice components for one or more crops. Yield values for example, represent grain yield. Other values such as biomass, pollen shed, silking can also be utilized. Training datasets can be used with various types of machine learning algorithms such as supervised learning, unsupervised learning, semi-supervised learning, and reinforcement learning. Neural network algorithm is an example of supervised learning—where a special purpose computer or a computing system is provided with training data containing the input/predictors along with the correct output. From the training data the computer/algorithm should be able to learn the patterns. Supervised learning algorithms model associations and dependencies between the target prediction output and the input features such that the output values for new data based on those previous associations that the network learned from. Training datasets can include measured data, simulated data, or a combination thereof.

In an embodiment, training data also includes for example, genetic associations for disease resistance and one or more of agronomic parameters such as planting density, pesticide application, nutrient inputs, water availability and one or more management practice data. Not each of the data types is needed to train the deep learning network.

Datasets may include data obtained from various crop field and/or greenhouse evaluations. These data include for example, geographical location, pest infestation patterns over a plurality of growing seasons, weather history, historical precipitation, GDU, soil type, soil moisture, soil temperature, management practices, and additional information such as for example, crop rotation, soil or seed-applied crop protection agents, applied nitrogen, cover crop presence or practice and other agronomically relevant parameters. Agricultural special purpose computer systems capable of monitoring, measuring and analyzing additional data from a plurality of agricultural fields are described herein. For example, such computers may receive one or more of such data either directly from the plurality of fields or evaluation stations or sensors or input by users.

### EXAMPLES

### Example 1: Phakopsora pachyrizi Asian Soybean Rust Monitoring and Treatment Protocol Recommendation

Phakopsora pachyrizi is the causative organism for Asian soybean rust (ASR), which infects soybean crops, causing losses amounting to billions of dollars worldwide. P. pachyrizi is a fungus of the order Urediniales, which fungi produce uredinia (fruiting bodies) that produce spores. In infected soy plants, the fungus produces brown lesions with uredinia on the underside of the plant leaflets. The uredinia detach from the plant after the leaf surface dries out. The spores, which are released from the uredinia through an ostiole, are lightweight and can be spread over long distances by air currents. On deposition onto a new host leaf, the fungus directly penetrates through the host cell walls and reproduction begins about one week after infection.

P. pachyrizi is subject to chemical control using fungicides of the DMI (demethylation inhibitor), class, including the triazoles and imidazoles, the QoI (strobiliurin) fungicide class, and the succinate dehydrogenase inhibitor (SDHI) class. Resistance to certain fungicides has developed and has been connected to point mutations in certain genes, such as the cyp51, sdhc, or cyto genes. Because of this resistance development, new tools and approaches are needed to develop an integrated disease management strategy.

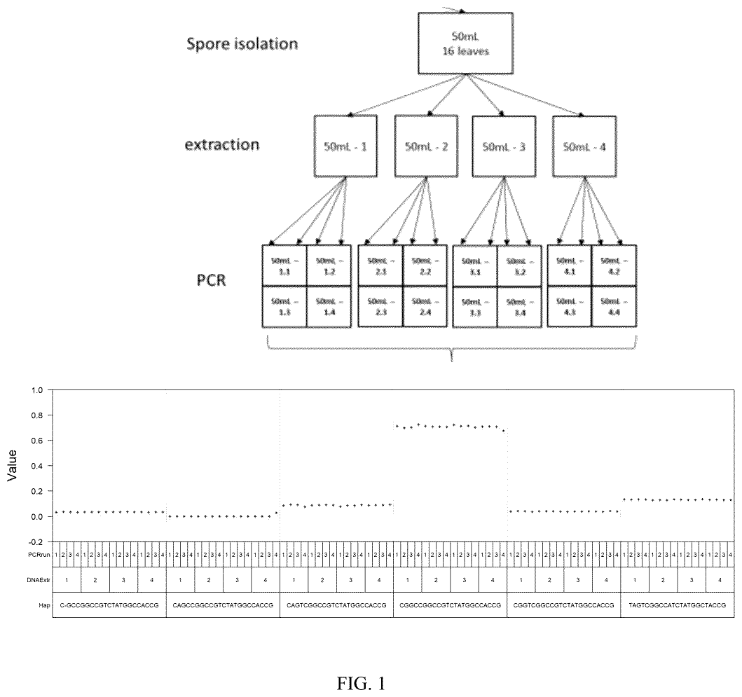

In this embodiment of the disclosure, ASR infected soy leaves are collected in a field and DNA is extracted from the infected leaves. In a next step, the ASR genes of interest (e.g. cyp51, sdhc, cytb) are sequenced. This can be done effectively as a bulk of multiple field samples, where each field sample is uniquely labeled, on a PacBio Sequel II. Genotypes are assessed per gene, where a genotype is based on one or more known relevant or unknown but frequent mutations/markers within the gene. By sequencing multiple targeted genes per field, the frequency of each genotype can be established for that field. In FIG. 1, it is shown that this frequency of genotypes in a sample can be established with high accuracy.

FIG. 1 depicts sampling of bulked leaves. A single field sample of 16 bulked leaves was split in four sub samples for DNA extraction, and subsequently each sub-sample was used in four independent targeted DNA multiplications steps each using PCR for the CYP51 gene. The 16 subsamples were each uniquely coded and subsequently sequenced. The frequency of different genotypes in each sub-sample was established. In this case there were 6 genotypes for the CYP51 gene. As can be seen, the frequency of each genotype is nearly identical for each of the 16 sub-samples, confirming the accuracy of the process to determine the relative genotype frequency of a field sample.

The selection of locations at which to collect spores may be influenced by previously-acquired remote sensing data that indicate the presence of P. pachyrizi or progression of the ASR disease. For each major genotype, one or more strains with that genotype are used for phenotyping assays to determine the pesticide resistance factor for different actives. If a resistance factor is not yet known for a (new) genotype, a sample is found with a large abundance of that genotype and a single spore isolation will be done on that sample to find the right genotype for subsequent phenotyping experiments.

From the frequency of the different genotypes at the sampling locations, information may be generated to predict the frequency of each genotype at a certain location. By combining this geographical information of the predicted local frequency of different genotypes with the resistance factor per genotype for each pesticide, a weighted pesticide resistance factor can be established for the local situation. In a preferred embodiment, some genotypes relate to differing alleles of the CYP51, SDHC, SDHB, SDHD, CYTB, OSBP (oxysterol binding protein inhibitor), and MDR (multidrug resistance) genes of P. pachyrizi. Some such genotypes of CYP51 are known (see, e.g., Mueller et al., Multiple resistance to DMI, QoI and SDHI fungicides in field isolates of Phakopsora pachyrhizi (Crop Protection, Volume 145 (July 2021). Some genotypes of SDHB, SDHC and SDHD are known to have a higher resistance to SDHI actives. Three important groups of fungicides are known to bind at three different sites within cytochrome b (CYTB), which is involved in complex III of the electron transport chain. The largest group is QoI, other groups are QoSI and QiI. Mutations of this gene are known to cause resistance to these fungicides; see, for example, “Relationship between QoIs, QoSIs and QiI Fungicides,” available at https://www.frac.info/docs/default-source/working-groups/qoi-quick-references/relationship-between-qii-qoi-and-qosi-fungicides.pdf?sfvrsn=c6db449a_2).

Such genotype information is then translated/combined to fungicide resistance information for different actives. The resistance information may be based on a weighted sum of the resistance factors for each genotype. In some embodiments, the remote sensing data may be employed in generating the geographical information or the resistance information. In a preferred embodiment, the sequencing of spores is based on kits or devices permitting rapid identification of the genotypes and thus identification of the localized pattern of resistance.

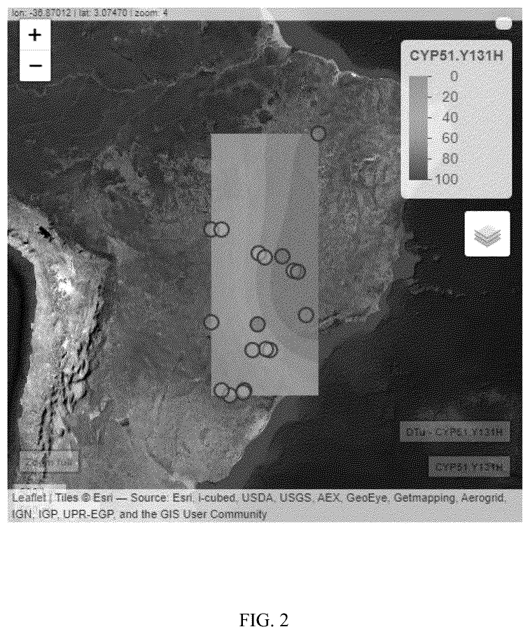

FIG. 2 shows a genotypic map that could be used to predict resistance to actives within the DMI group, clearly showing a gradient for increased resistance in Brazil.

The resistance information may then be employed to generate automated spray recommendations. These recommendations may relate to the selection of the fungicidal agent, the amount to be sprayed, the timing of the spray, specific locations within fields to be sprayed, or any combination thereof. In some embodiments, the spray recommendation may be provided in near-real time, contemporaneously with the sequencing of the spores.

In some embodiments, historical spray data for each location may be used to track progression of the ASR disease and efficacy of past treatments. In some cases, comparison of such historical spray data with currently acquired data may lead to a revision of the resistance information.

### Example 2: Management of Septoria glycines in Soy

A further embodiment of the present disclosure relates to the management of Septoria brown spot (Septoria glycines) in soy crop fields. Septoria brown spot is one of the most common fungal foliar diseases in soybean. The usual symptoms of the disease are brown lesions surrounded by an area displaying chlorosis. The disease is transmitted via the formation of pycnidiaspores formed in the overwintering crop residue, which are spread by rain to the low canopy of the subsequent soy crop. The disease is generally controlled by use of fungicides from one of several groups: the QoI stobilurins such as azoxystrobin (although resistant strains are developing), the DMI triazoles such as cyproconazole, and the SDHI carboxamindes such as boscalid. Similarly to the embodiment described above, sequencing data can be obtained for the spores and resistance maps developed based on known resistance alleles and others developed as sequencing and spore resistance phenotyping data is compiled, and targeted fungicide application recommendations can be made.

1-143. (canceled)

144. A method of generating a pesticide resistance map of a target pest, the method comprising:
(a) obtaining genotypic information from a plurality of pest samples obtained from a plurality of soy field locations, wherein the pest samples comprise fungal material obtained from either Phakopsora pacyrizi or Septoria glycines;
(b) generating a frequency of one or more genotypes based on the genotypic information, wherein a correlation exists between the one or more genotypes and resistance to at least one pesticide, wherein the correlation is quantified as at least one pesticide resistance factor to at least one pesticide;
(c) correlating the one or more genotypes to the plurality of soy field locations to generate a genotype frequency map;
(d) generating a pesticide resistance map based on the genotype frequency map and the pesticide resistance factor of each genotype;
(e) identifying candidate pesticides for use in a pesticide application protocol based on the pesticide resistance map; and
(f) generating the pesticide application protocol for a target soy field location using the pesticide resistance map.

145. The method of claim 144, additionally comprising:
(g) selecting, based on the pesticide resistance map, a plurality of validating assays for the pesticide application protocol; and
(h) performing the validating assays to generate a validated pesticide application protocol.

146. The method of claim 144, wherein the genotypic information is obtained at a location remote from the plurality of soy field locations.

147. The method of claim 144, wherein the genotypic information is obtained in the plurality of soy field locations.

148. The method of claim 144, wherein obtaining genotypic information is conducted contemporaneously with obtaining pest samples.

149. The method of claim 145, wherein the validating assays are performed in the plurality of soy field locations.

150. The method of claim 144, wherein the correlation between the one or more genotypes and resistance to at least one pesticide is based on data from at least one previous season.

151. The method of claim 145, wherein pre-existing data for the plurality of soy field locations are employed in generating the pesticide application protocol.

152. The method of claim 151, additionally comprising:
(f.i) comparing the obtained genotypic information to historic or pre-existing genotypic information for the location; and
(f.ii) identifying changes in pesticide resistance based on the results of the comparison;
wherein generating the validated pesticide application protocol is based on the pesticide resistance factors and the changes in pesticide resistance.

153. The method of claim 144, additionally comprising, before step (e):
(d.i) obtaining a plurality of spectral images of the plurality of soy field locations; and
(d.ii) identifying a plurality of localized disease states based on the plurality of spectral images;
and wherein generating the pesticide resistance map additionally comprises correlating the plurality of localized disease states with the genotype frequency map.

154. The method of claim 153, wherein:
obtaining a plurality of spectral images of the plurality of soy field locations comprises monitoring an unmanned aerial vehicle (UAV) as the UAV flies along a flight path above the plurality of soy field locations and as the UAV performs:
(i) capturing a plurality of images of the plurality of soy field locations as the UAV flies along the flight path; and
(ii) transmitting the plurality of images to an image recipient.

155. The method of claim 153, wherein:
obtaining a plurality of spectral images of the plurality of soy field locations comprises
obtaining a plurality of satellite-generated images of the soy field locations.

156. The method of claim 144, wherein the pesticide application protocol comprises a recommended pesticide and a recommended application timing.

157. The method of claim 144, wherein the plurality of pest samples are obtained from the air, soil, water, plant part or a combination thereof.

158. The method of claim 157, wherein the plurality of pest samples are fungal material selected from the group consisting of mycelium or spores.

159. The method of claim 144, wherein the one or more genotypes are generated by testing for alleles of genes involved in resistance to pesticides, and wherein the alleles are alleles selected from the group consisting of the CYP51, SDHC, SDHB, SDHD, CYTB, OSBP and multi-drug resistance genes.

160. The method of claim 16, wherein the alleles selected from the group consisting of the CYP51, SDHC, SDHB, SDHD, CYTB, OSBP and multi-drug resistance genes are correlated with the resistance to at least one pesticide to develop the at least one pesticide resistance factor to at least one pesticide.

161. The method of claim 160, wherein the at least one pesticide belongs to a class of pesticides selected from the group consisting of fungicides, nematicides, bactericides, and insecticides.

162. The method of claim 144, wherein the at least one pesticide comprises a triazole fungicide selected from the group consisting of cyproconazole, propiconazole, tebuconazole, myclobutanil, epoxiconazole, triadimenol, prothioconazole, metconazole, flusilazole, paclobutrazol, and tetraconazole.

163. The method of claim 162, wherein the at least one pesticide additionally comprises a strobilurin fungicide selected from the group consisting of fluoxastrobin, mandestrobin, pyribencarb, azoxystrobin, coumoxystrobin, enoxastrobin, flufenoxystrobin, picoxystrobin, pyraoxystrobin, pyraclostrobin, pyrametostrobin, triclopyricarb, dimoxystrobin, fenaminstrobin, metominostrobin, orysastrobin, kresoxim-methyl, trifloxystrobin, fenamidone, and famoxadone.
